(19) United States
(12) Patent Application Publication
(10) Pub. No.: US 2023/0321429 A1
(43) Pub. Date: Oct. 12, 2023

(54) Device, System, and Method for Treating Restless Leg Syndrome

(71) Applicant: Candy Oxenrider, Pittsburgh, PA (US)

(72) Inventor: Candy Oxenrider, Pittsburgh, PA (US)

(21) Appl. No.: 18/133,211

(22) Filed: Apr. 11, 2023

Related U.S. Application Data
(60) Provisional application No. 63/330,066, filed on Apr. 12, 2022.

(51) Int. Cl. A61N 1/04; A61N 1/36

(52) U.S. Cl. CPC A61N 1/0484; A61N 1/0456; A61N 1/36034

(57) ABSTRACT

A device and method for treating, for example the Restless Leg Syndrome, an individual comprising a pair of leggings, at least one electrode, the at least one electrode attached to the pair of leggings, and a pulse generator, the pulse generator connected to the at least one electrode.

### CROSS-REFERENCE TO RELATED APPLICATIONS

The present application claims priority to U.S. Provisional Patent Application No. 63/330,066, filed Apr. 12, 2022, the content of which is incorporated herein by reference in its entirety.

### BACKGROUND OF THE INVENTION

### Field of the Invention

This disclosure relates generally to devices, systems, and methods treating an individual with electric pulses, and, in particular, to devices, systems, and methods for treating Restless Leg Syndrome (RLS) and/or symptoms associated with RLS.

### Technical Considerations

Restless Leg Syndrome (RLS), also known as Willis-Ekborn disease, is a condition that causes an urge to move the legs. The urge may be caused by an uncomfortable sensation. The urge is known to occur often during nighttime while a patient is resting or sleeping, which can result in poor sleep, excessive daytime sleepiness, and interference with daily activities. The urge is lessened by movement such as stretching, kicking, pacing, walking, and/or like movement of the legs. Treatments for RLS may include medications, such as sleep medication, muscle relaxants, opioids, etc. Treatments may also include natural remedies such as massages, baths, exercise, and avoiding stimulants. However, such treatments may not be desirable for individuals who do not want to take, particularly on a long-term basis, opiate medications or medications that, for example, alter dopamine metabolism. Accordingly, there is a need in the art for new devices, systems, and methods for treating RLS and symptoms associated with RLS.

### SUMMARY OF THE INVENTION

According to some non-limiting embodiments, a device for treating individuals may comprise a pair of pants including at least one electrode, the at least one electrode attached to the pair of pants, a pulse generator, and a power source, the pulse generator and power source connected to the at least one electrode. The device may be configured to produce electric pulses at a frequency between 0-120 Hz, a pulse width of between 0-500 μs, and varying intensity. The device may further comprise a remote control, wherein the remote control is configured to control frequency, pulse width, and intensity of the at least one electrode.

According to some non-limiting embodiment, a method of treating an individual is provided, the method comprising: placing at least one electrode on the individual; and providing an electric pulse to the at least one electrode. The method may further comprise: varying a frequency of the electric pulse between 0-120 Hz, varying a pulse width of the electric pulse between 0-500 μs, and varying an intensity of the electric pulses. Such method may stimulate human skin by electrode current to help the individual increase muscle, reduce fat, lose weight, keep fit, promote metabolism, increase strength, relieve pressure, enhance immunity, improve sleep, and relieve muscle atrophy.

### BRIEF DESCRIPTION OF THE DRAWINGS

FIGS. 1A-1B show non-limiting aspects of a garment;

FIGS. 2A-2C show non-limiting aspects of a garment;

FIGS. 3A-3C show non-limiting aspects of the garment as shown in FIGS. 1a-1b;

FIG. 4A-4C show non-limiting aspects of a control device of the garment; and

FIG. 5 shows a diagram of a non-limiting embodiment of components of one or more devices of FIGS. 4A-4C.

### DESCRIPTION OF THE INVENTION

As used herein, the singular forms of “a”, “an”, and “the” include plural referents unless the context clearly dictates otherwise.

For the purposes of this specification, unless otherwise indicated, all numbers expressing quantities, dimensions, physical characteristics, and so forth are to be understood as being modified in all instances by the term “about”. Unless indicated to the contrary, the numerical parameters set forth in the following specification and attached claims are approximations that can vary depending upon the desired properties sought to be obtained by the present invention.

Notwithstanding that the numerical ranges and parameters setting forth the broad scope of the invention are approximations. Any numerical value inherently contains certain errors. It should be understood that any numerical range recited herein is intended to include all sub-ranges subsumed therein. For example, a range of “1 to 10” is intended to include many and all sub-ranges between and including the recited minimum value of 1 and the recited maximum value of 10; that is, all subranges beginning with a minimum value equal to or greater than 1 and ending with a maximum value equal to or less than 10, and all subranges in between, e.g., 1 to 6.3, or 5.5 to 10, or 2.7 to 6.1.

As used herein, the terms “communication” and “communicate” refer to the receipt, transmission, or transfer of one or more signals, messages, commands, or other type of data. For one unit or device to be in communication with another unit or device means that the one unit or device is able to receive data from and/or transmit data to the other unit or device. A communication can use a direct or indirect connection, and can be wired and/or wireless in nature. Additionally, two units or devices can be in communication with each other even though the data transmitted can be modified, processed, routed, etc., between the first and second unit or device. For example, a first unit can be in communication with a second unit even though the first unit passively receives data and does not actively transmit data to the second unit. As another example, a first unit can be in communication with a second unit if an intermediary unit processes data from one unit and transmits processed data to the second unit. It will be appreciated that numerous other arrangements are possible. Any known electronic communication protocols and/or algorithms can be used such as, for example, TCP/IP (including HTTP and other protocols), WLAN (including 802.11a/b/g/n and other radio frequency-based protocols and methods), analog transmissions, Global System for Mobile Communications (GSM), 3G/4G/LTE, BLUETOOTH, ZigBee, EnOcean, TransferJet, Wireless USB, and the like known to those of skill in the art.

As used herein, “electrical communication,” for example in the context of transmitting electrical pulses from a pulse generator to an electrode refers to sending an electrical pulse produced by a pulse generator to a skin surface electrode or like devices capable of generating electrical current, or converting electrical current into a mechanical stimulation, to stimulate a patient, for example a patient's skin, muscles, nerves, and/or neurons, as described herein.

The “intensity” of an electrical pulse is proportional to, and refers to the voltage and/or current (e.g., milliAmperes or mA) applied to patient, with an increased intensity being proportional to an increased voltage or an increased current applied to the nerve or neuron.

It should be recognized that the optimal electrical stimulation parameters to elicit a desired effect may vary to some degree from subject-to-subject, depending on a number of factors. Optimal frequencies to elicit the desired goals can be adjusted from person-to-person. A “patient” may be human or animal and unless specified otherwise embraces a specific patient, a class of patients, or any human or animal in a generic sense and does not imply any doctor-patient relationship. Thus, a structure configured to, or adapted to, a patient's limb or core structure includes structures configured to a specific patient and/or a group of patients.

The electrical stimulation described herein can include electrical pulses that can have any suitable characteristic, so long as the stimulation is effective to achieve the desired physiological response. As such, the terms “electrical stimulation” and “electrical pulses” are used interchangeably herein. As will be recognized by a person of skill in the art, characteristics of the electrical pulses, including, without limitation, amplitude (pulse strength, referring to the magnitude or size of a signal voltage or current), voltage, amperage, duration (e.g., pulse width), frequency, polarity, phase, relative timing, and symmetry of positive and negative pulses in biphasic stimulation, and/or wave shape (e.g., square, sine, triangle, sawtooth, or variations or combinations thereof) may be varied in order to provide the desired result in a patient or class of patients. So long as other characteristics of the electrical signals (e.g., without limitation, amplitude, voltage, amperage, duration, polarity, phase, relative timing and symmetry of positive and negative pulses in biphasic stimulation, and/or wave shape) are within useful ranges, modulation of the pulse frequency will achieve the desired result.

One characteristic of the electrical signals used to produce a desired response, as described above, is the frequency of the electrical pulse. Although effective ranges (e.g., frequencies able to produce a stated effect) may vary from subject-to-subject, and the controlling factor is achieving a desired outcome, certain, non-limiting exemplary ranges may be as follows. In aspects, useful frequencies range from about 1 Hz (Hertz) to about 120 Hz, all values and subranges therebetween inclusive. For example, at a low frequency, for example between 1-20 Hz, a weak muscle twitch or flutter may be generated. As the frequency increases, the strength of the contraction in the muscle increases. At low frequencies, muscles can be controlled by the user, thereby allowing the user to further stimulate the muscles through exercise. At a frequency higher range, for example 60-120 Hz, however, the electrical stimulation may impact the sensory neurons than the motor neurons, thereby decreasing the correlation between muscle contraction and the frequency at higher ranges.

As indicated above, a characteristic of electrical pulses is their intensity which in a medium of stable or relatively stable resistance, such as mammalian tissue, can be characterized as relating to current (I, typically measured in mA), or voltage (V, typically measured in mV or V), based on Ohm's Law. It should, therefore, be understood that the intensity of the stimulation is a matter of both V and I, and as such, both are increased, e.g., proportionally or substantially proportionally, with increased intensity of stimulation. As such, one characteristic of the pulses is the current that is applied to produce a physiological response. Stimulation can be achieved in a typical range of from 0.01 mA to 10 mA, all subranges and values therebetween inclusive.

Another characteristic of the intensity of the pulses is voltage. Stimulation can be achieved in a typical range of from 1 mV to 50 V, all subranges and values therebetween inclusive.

As indicated above, the waveform of the pulses may vary, so long as the desired physiological response is realized. One skilled in the art will appreciate that other types of electrical stimulation may also be used in accordance with the present invention. Monophasic or biphasic stimuli, or a mixture thereof, may be used. Damage to the nerve(s) by the application of an electrical current may be minimized, as is known in the art, by application of biphasic pulses or biphasic waveforms to the nerve(s), as opposed to monophasic pulses or waveforms that can damage nerves in some instances of long-term use. “Biphasic current”, “biphasic pulses”, or “biphasic waveforms” refer to two or more pulses that are of opposite polarity that may be of equal or substantially equal net charge (hence, biphasic and charge balanced), and may be symmetrical, asymmetrical, or substantially symmetrical. This is accomplished, for example, by applying through an electrode one or more positive pulses, followed by one or more negative pulses, typically of the same amplitude and duration as the positive pulses, or vice versa, such that the net charge applied to the target of the electrode is zero, or approximately zero. For charge-balanced biphasic stimulation, the opposite polarity pulses may have different amplitudes, profiles, or durations, so long as the net applied charge by the biphasic pulse pair (the combination of the positive and negative pulses) is approximately zero.

Stimulation can be delivered as needed, for a duration suitable to provide relief from symptoms of RLS. For example, and without limitation, in aspects of the method, the patient is stimulated for 1, 2, 3, 4, 5, 6, 7, 8, 9, 10, 11, 12, 13, 14, 15, 16, 17, 18, 19, 20, 21, 22, 23, and/or 24 hours per day, all values and subranges therebetween inclusive. In some aspects, the patient is stimulated continuously, for 24 hours. In some aspects, the patient is stimulated only during waking hours. In some aspects, the patient is stimulated for a period of time, followed by a period of equal or different duration where no stimulation is applied, and the cycle is repeated one or more times, for example 5 times, 10 time, 20 times, or more, all values and subranges therebetween inclusive.

As described above, the stimulation may be applied intermittently (that is, the pulses are turned on and off alternately during a stimulation interval for any time period) during continuous or interval stimulation protocols. For example, the stimulation may be applied for 5 seconds on and 5 seconds off over an interval of, for example, 1-10 minutes or longer (e.g., hours, days, weeks, months, years). Other examples of intermittent application of pulses may be 1-90 seconds on and 1-90 seconds off over up to a 360 minute time period, all values and subranges therebetween inclusive. So long as other pulse parameters are within acceptable limits, the inhibition is temporary and does not damage the involved neurons/nerves. For example, intermittent application of pulses may be continuous, that is, for as long as the pulses are having the desired effect, and for as long as the patient desires (i.e., is not painful or harmful to the patient). In one aspect, the stimulation is provided continuously, for example, to treat severe symptoms, or any symptom that does not respond to intermittent, short-term stimulation to the degree desired by a clinician or the patient.

The waveform of the pulses of the stimulation may vary so long as the desired effect is realized. Examples of suitable waveforms include sine, square, rectangular, triangle sawtooth, rectilinear, pulse, exponential, truncated exponential, and damped sinusoidal. One skilled in the art will appreciate that other types of electrical stimulation may also be used in accordance with the device, system and methods described herein. Monophasic or biphasic stimuli, or a mixture thereof may be used. Damage to nerves by the application of an electrical current may be minimized, as is known in the art, by application of biphasic pulses or biphasic waveforms to the nerve(s), as opposed to a monophasic pulses or waveforms that can damage nerves in some instances of long-term use. “Biphasic current”, “biphasic pulses”, or “biphasic waveforms” refer to two or more pulses that are of opposite polarity that typically are of equal or substantially equal net charge (hence, biphasic and charge balanced) and may be symmetrical asymmetrical or substantially symmetrical. This is accomplished, for example, by applying, via an electrode, one or more positive pulses, followed by one or more negative pulses, typically of the same amplitude and duration as the positive pulses, or vice versa, such that the net charge applied to the target of the electrode is zero or approximately zero. The opposite polarity pulses may have different amplitudes, profiles or durations, so long as the net applied charge by the biphasic pulse pair (the combination of the positive and negative pulses) is approximately zero.

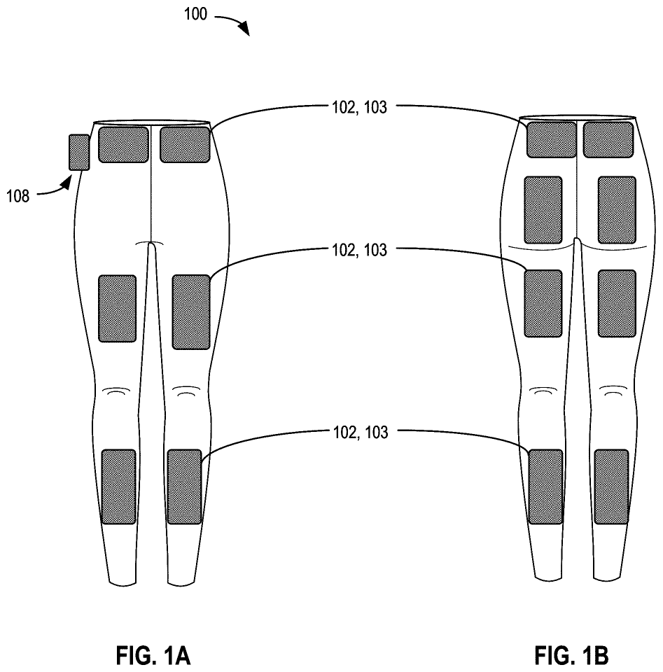

Referring to FIGS. 1A-1B, in some non-limiting embodiments of a garment 100 are shown. The garment 100 may be any clothing article that a patient can put on, such as trousers, leggings, shorts, compression pants, compression shorts, shirts, compression shirts, coats, jackets, vests, bands, etc. Garment 100 may vary in size, length, width, thickness, etc. and may be made of any material that can be worn on an individual. In the embodiments as shown in FIGS. 1A-1B, the garment 100 may be a pair of leggings. According to some non-limiting aspect, FIG. 1A shows a front view of the leggings and FIG. 1B shows a back view of the leggings. The leggings may include at least one electronic muscle stimulation (EMS) device, through pad 102. Alternatively and/or in addition, the trousers may include at least one mechanical stimulator 103. Mechanical stimulators may convert electrical signals to, for example, vibrations that may be applied to a wearer's skin. In some non-limiting embodiments, the electrical stimulation is not delivered with a transcutaneous electrical nerve stimulation (TENS) device. Those of skill will appreciate the differences between a TENS device and stimulation delivered via EMS, which at least includes that EMS is of greater intensity, causing muscle contractions.

According to some non-limiting embodiments, the EMS pad 102 may emit electrical pulses to the patient. The EMS pad 102 may vary in size, weight, and dimension. The EMS pad 102 may be made of any suitable material for emitting electric pulses. The EMS pad 102 may be made of a flexible material such that the individual may wear the garment 100 comfortably. According to some non-limiting embodiments, the device for treating individuals may comprise of a plurality of EMS pads 102. The EMS pads 102 may be placed anywhere on the device for treating individuals. The EMS pads 102 may be placed in specific locations such that there would be contact with muscle groups of the individual. Still referring to FIGS. 1A-1B, for instance, the EMS pads 102 may be placed around the waist, thighs, calves, shins, buttocks, hamstrings, etc. The EMS pads 102 and/or mechanical stimulators 103 may comprise electrodes and/or transducers in electrical communication with a pulse generator.

According to further non-limiting embodiments, mechanical stimulator 103 may be placed instead of and/or in addition to the EMS pad 102. The mechanical stimulator 103 may provide mechanical or vibrational force to the applied area. Alternatively, the EMS pad 102 and mechanical stimulator 103 may be placed throughout the garment 100 such that the effective area of the EMS pad 102 and mechanical stimulator 103 do not overlap. According to some non-limiting embodiments, the EMS pad 102 and mechanical stimulator 103 may be placed throughout the garment 100 such that the effective area of the EMS pad 102 and mechanical stimulator 103 overlap. In non-limiting embodiments, EMS pads 102 and/or mechanical stimulators 103 are arranged on an inner surface of garment 100, such that they can contact, or are arranged in close proximity to, a patient's skin. The EMS pads 102 and/or mechanical stimulators 103 may be in wired or wireless communication with a control device 108. The control device may be programmed to control the EMS pads 102 and/or mechanical stimulators 103. The control device 108 may be attached to the garment 100, or alternatively, the control device 108 may be remote from the garment 100.

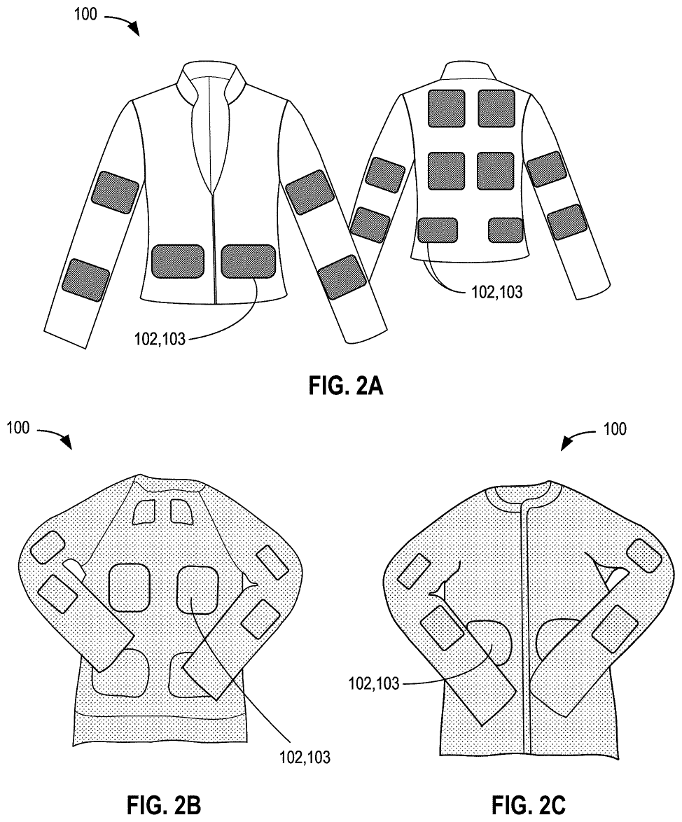

Referring to FIGS. 2A-2C, shown are some non-limiting embodiments of the garment 100 in the form of a jacket. The EMS pads 102 and/or mechanical stimulators 103 may be placed in any location throughout the jacket. The EMS pads 102 and/or mechanical stimulators 103 may be placed in specific locations such that there would be contact with muscle groups of the individual, such as the abs, upper arm, lower arm, shoulders, upper back, back, lower back, etc. The EMS pads 102 and/or mechanical stimulators 103 may be attached onto the garment 100. For instance, the EMS pads 102 and/or mechanical stimulators 103 may be sown on, attached via hook and loop fasteners, placed in pockets of the garment 100 configured to receive the EMS pads 102 and/or mechanical stimulators 103, glued on, etc. In non-limiting embodiments, EMS pads 102 and/or mechanical stimulators 103 are arranged on an inner surface of garment 100, such that they can contact, or are arranged in close proximity to, a patient's skin.

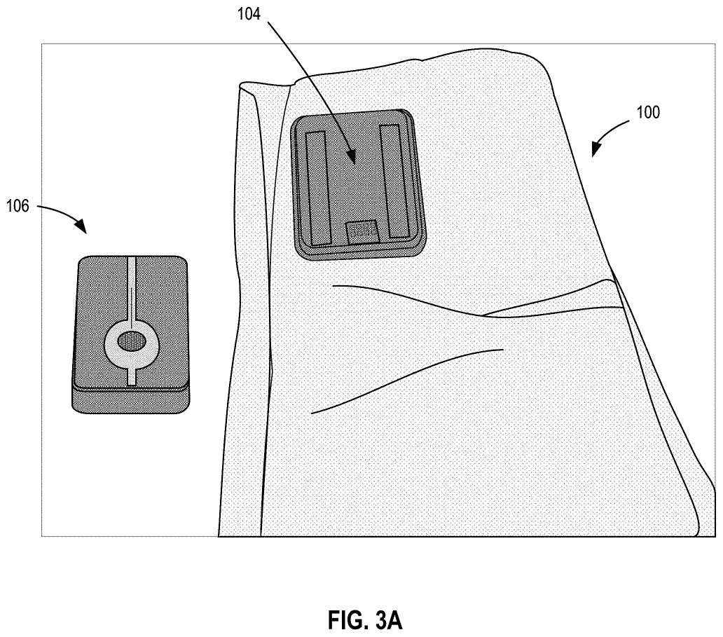

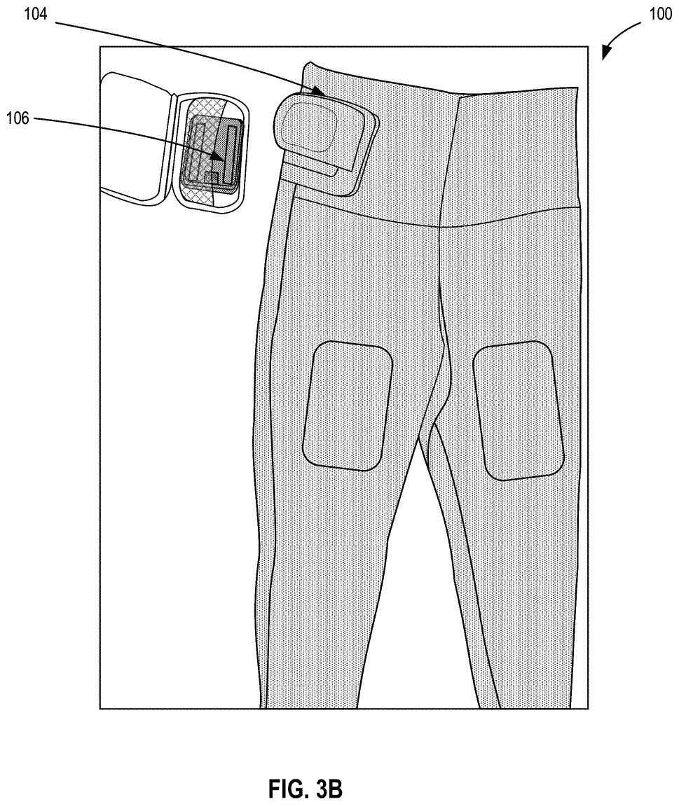

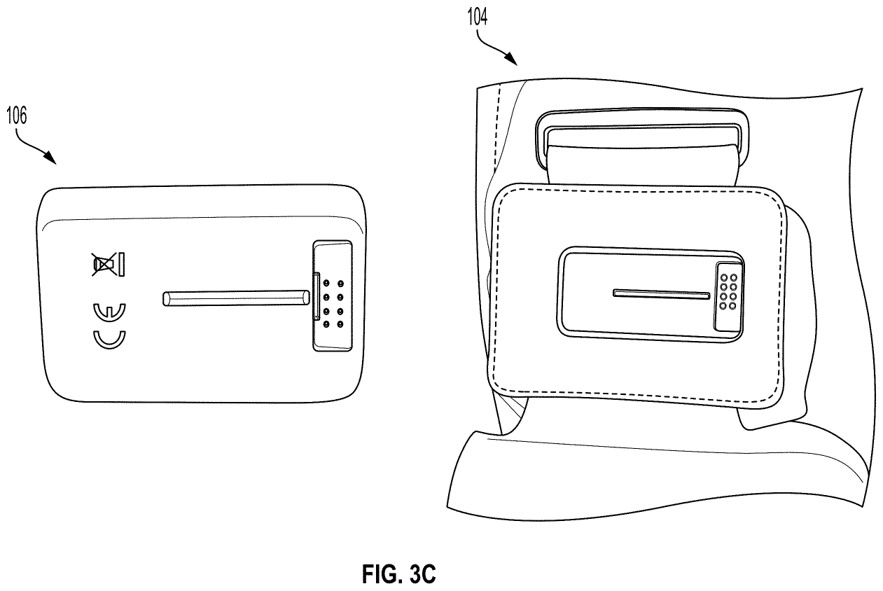

Referring to FIGS. 3A-3C, shown are some non-limiting embodiments of the garment 100 in the form of leggings as shown in FIGS. 1A-1B. According to some non-limiting embodiments, the device for treating individuals may have a power source 106. The power source 106 may be configured to provide electricity to the EMS pads 102 and/or mechanical stimulators 103 to allow the EMS pads 102 to produce electric pulse and to allow the mechanical stimulators 103 to produce mechanical or vibrational force. The power source 106 may be in electrical communication with pulse generator of EMS pads 102. There may be one or more power source 106. The power source 106 may be removably attached to the garment 100 such that the garment 100 may further include power source adapter 104. The power source adapter 104 may be configured to receive the power source 106 and secure the power source 106 in place. For example, FIG. 3A shows the power source adapter 104 having two grooves that correspond with the power source 106. In another example, FIG. 3C shows the power source adapter 104 having one groove that correspond with a protrusion of the power source 106 that allow the power source adapter 104 and the power source 106 to be removably attached to the garment 100. The power source 106 may be made of any material suitable to provide electricity to the pulse generator(s). The power source 106 may be rechargeable or may be compatible with commonly sold batteries. The power source 106 and the power source adapter 104 may be attached to the garment 100 in any suitable means, such as sown on, glued, etc. Alternatively, the garment 100 may be configured to connect to power sources such as power outlets, such that EMS 102 pads can be powered via a non-remote power source.

With continuing reference to the figures, garment 100 may further include a pulse generator, as described above, for generating pulses through EMS pads 102 and/or actuating mechanical stimulators 103. Those of skill in the art will appreciate that pulse generators may be programmed, for example through instructions stored in memory and executed by one or more processors as described below, to deliver stimulation as described above.

Still referring to FIGS. 3A-3C, the power source 106 may be configured to provide enough electricity to one or more pulse generators to provide sufficient stimulation through all of the EMS pads 102 and/or mechanical stimulators 103. The EMS pads 102 may be configured to provide electric pulses with varying characteristics. For instance, the EMS pads 102 may be configured to produce electric pulses with frequency between 0-1000 Hz, 0-500 Hz, or 0-120 Hz, all values and subranges therebetween inclusive. The EMS pads 102 may be configured to produce electric pulses with pulse width between 0-2000 μs, 0-1000 μs, or 0-500 μs or 50-500 μs, all values and subranges therebetween inclusive. According to some non-limiting embodiments, the force, intensity, rate, duration, etc. of the vibrational force of mechanical stimulators 103 may vary and be adjusted.

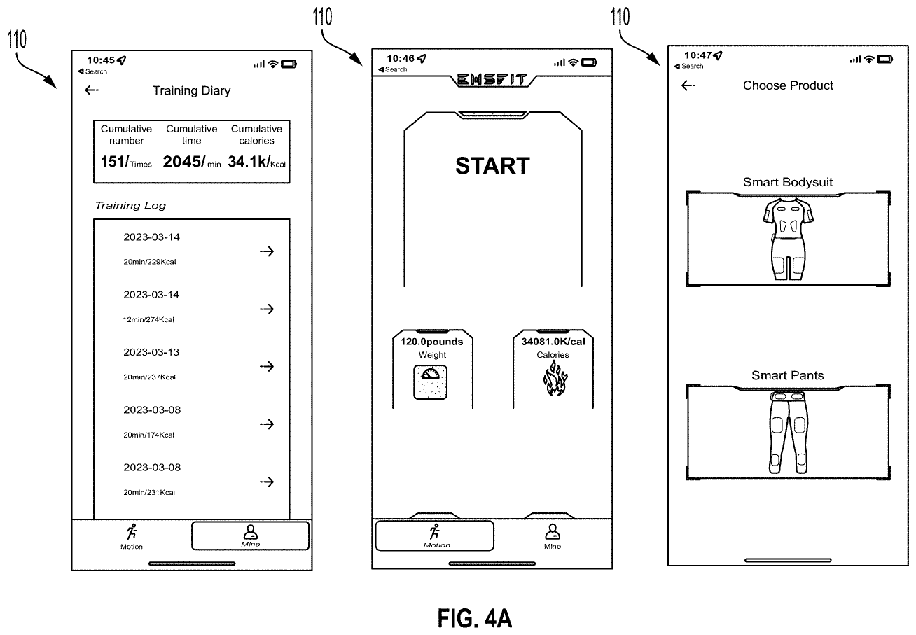

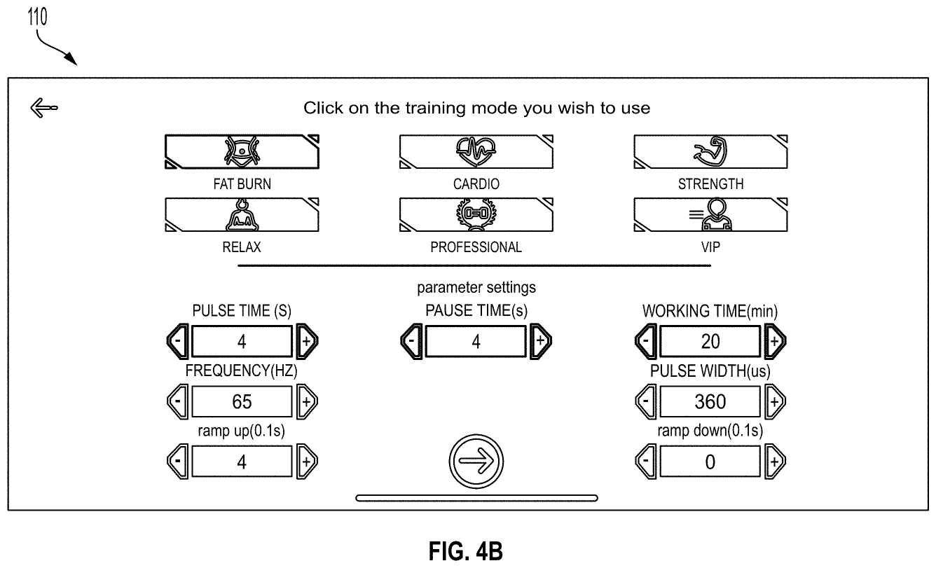

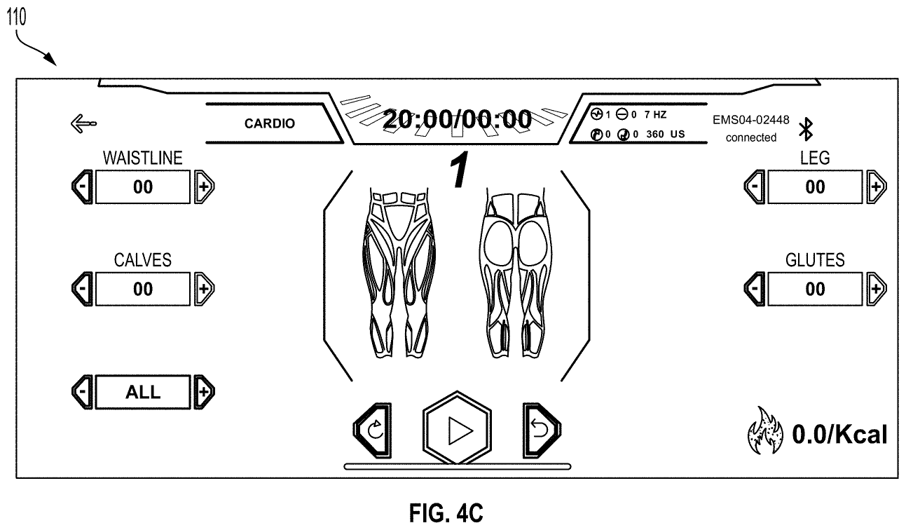

Referring to FIGS. 4A-4C, shown is a non-limiting embodiment of a control device 108. The control device 108 may be an app, a phone, a remote controller, a software on a computer, etc. According to a non-limiting embodiment, FIG. 4A shows the control device 108 as a mobile device having an application thereon. As used herein, the term “mobile device” may refer to one or more portable electronic devices that are configured to communicate with one or more networks. As an example, a mobile device may include a cellular phone (e.g., a smartphone or standard cellular phone), a portable computer, a wearable device (e.g., watches, glasses, lenses, clothing, and/or the like), a PDA, and/or other like devices.

A patient may interact with the application of the control device 108 to control numerous functional aspects of the garment 100. A mobile device may be able to remotely control the device for treating individuals by numerous means, such as Bluetooth connection, wired connections, WiFi, near-field communication, and other known communication methods. Via the control device 108, a use may control the upper garment 100, lower garment 100, and/or both garments 100. The control device 108 may be configured to track and show previous uses of the garment 100. For example, the control device may track and display the number of calories expended at specific times and/or over a certain period of time by the patient. The control device may track and display the amount of times and the number of times the garment 100 was used by the patient. The control device 108 may provide a wide range of information pertinent to the particular use purpose of the individual, such as weight loss, rehabilitation, muscle gain, etc. Referring to FIGS. 4B and 4C, the control device 108 may dictate the physical characteristics of each of the EMS pads 102 and/or mechanical stimulators 103. For instance, the control device 108 may allow a patient to, or through feedback provided by one or more sensors may allow the device and/or application to, control the frequency, pulse width, pulse duration, interstimulus interval, intensity, and/or duration of stimulation from of each of the EMS pads 102 and/or the mechanical or vibrational forces emitted from mechanical stimulators 103. In non-limiting embodiments, control device 108 may allow a patient to execute a ramp of stimulation, that is, stimulation may increase or decrease by a desired amount over a desired period of time (e.g., a patient may set control device 108 to increase the frequency by 10 Hz every 30 seconds). The control device 108 may be used to change the physical characteristics of each of the EMS pads 102 depending on the patient's reason for using the garment 100, such as fat burn, cardio, strength training, relaxing, treating cellulite, treating one or more symptoms of RLS, and the like. As used herein, “one or more symptoms of RLS” refers to an urge to move and/or discomfort in the limbs (e.g., aching, electric, throbbing, pulling, itching, crawling, and/or creeping). In non-limiting embodiments, exemplary parameters for treating symptoms of RLS involve stimulation at about 0 Hz to 120 Hz, 5 Hz to about 100 Hz, optionally about 5 Hz to about 85 Hz, optionally about 5 Hz to about 7 Hz, optionally about 7 Hz, all subranges and values therebetween inclusive, with a pulse width of about 200 μs to about 500 μs, optionally about 300 μs to about 400 μs, optionally about 350 μs, optionally about 360 μs, all subranges and values therebetween inclusive, and a pulse duration of about 1 second to about 3 seconds, optionally about 1 second, optionally 1 second on, 1 second off, optionally two seconds on two seconds off, optionally continuously, all subranges and values therebetween inclusive. In non-limiting embodiments, a ramp of stimulation may occur in about 0.1 to about 5 second intervals, optionally about 0.1 to about 3 second intervals, optionally about 0.1 to about 0.4 second intervals, all values and subranges therebetween inclusive. In non-limiting embodiments, such stimulation may be delivered for as long as relief is received, optionally 20 minutes to one hour, optionally about 20 minutes to about 40 minutes, optionally about 25 minutes to about 30 minutes, all subranges and values therebetween inclusive. In non-limiting embodiments the same stimulation is delivered to one or more, optionally to each, EMS pad 102 and/or mechanical stimulator 103. In non-limiting embodiments, different stimulation is delivered to at least one EMS pad 102 and/or mechanical stimulator 103. For example, 7 Hz and 350 μs may be used for cardio training to improve cardiopulmonary capacity, as well as to eliminate accumulated metabolites and improve blood circulation. The control device 108 may allow the user to vary the frequency, the pulse width, and the time trained. For ease of use, the control device 108 may indicate the minimum and the maximum frequency and the pulse width capabilities with an arbitrary number range, such as 0 to 100 and 0 to 10. For example, 0 Hz may be represented by number 0 and 120 Hz may be represented by number 100. Pulse width of 0 μs may be represented by 0 and 500 μs may be represented by 100. For treating RLS, relative strength of 55 may be used for waist, leg, and glutes, and 75 may be sued for calves. It is appreciated any range of frequency and pulse width may be programmed to be controlled by the arbitrary number range for ease of use.

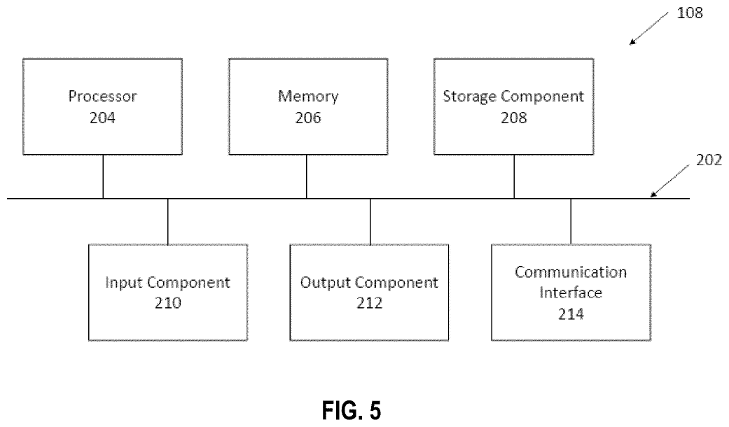

Referring now to FIG. 5, FIG. 5 is a diagram of example components of a control device 108, which as noted above may be a mobile device. As shown in FIG. 5, control device 108 may include a bus 202, a processor 204, memory 206, a storage component 208, an input component 210, an output component 212, and a communication interface 214.

Bus 202 may include a component that permits communication among the components of device 200. In some non-limiting embodiments, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of control device 108. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits control device 108 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from control device 108 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit control device 108 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Control device 108 may perform one or more processes described herein. Control device 108 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software.

The number and arrangement of components shown in FIG. 5 are provided as an example. In some non-limiting embodiments, control device 108 may include additional components, fewer components, different components, or differently arranged components than those shown in FIG. 5. Additionally, or alternatively, a set of components (e.g., one or more components) of control device 108 may perform one or more functions described as being performed by another set of components of control device 108.

While several embodiments of garment 100 are configured to provide electric pulses were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. A garment for treating restless leg syndrome comprising:
an article of clothing;
at least one electrode, the at least one electrode attached to the article of clothing; and
a pulse generator, the pulse generator connected to the at least one electrode.

2. The device of claim 1, wherein the at least one electrode produces electric pulses at a frequency between 0-120 Hz.

3. The device of claim 1, wherein the at least one electrode produces electric pulses, the electric pulses having a pulse width of between 0-500 μs.

4. The device of any of claim 1, wherein the at least one electrode produces electric pulses having a varying intensity.

5. The device of claim 1, further comprising a remote control, wherein the remote control is configured to control frequency, pulse width, and/or intensity of pulses delivered through the at least one electrode.

6. The device of claim 1, further comprising a power source.

7. The device of claim 1, further comprising a mechanical stimulator.

8. The device of claim 1, wherein the article of clothing is a pair of leggings.

9. The device of claim 1, wherein the article of clothing is a shirt or jacket.

10. A method of treating at least one symptoms of restless leg syndrome (RLS) in a patient comprising:
arranging the device of claim 1 on the patient; and
providing at least one electric pulse through the at least one electrode, thereby treating the at least one symptom of RLS.

11. The method of claim 10, further comprising: varying a frequency of the electric pulse between 0-120 Hz.

12. The method of claim 101, further comprising: varying a pulse width of the electric pulse between 0-500 μs.

13. The method of claim 10, further comprising varying an intensity of the electric pulse.

14. The method of claim 10, further comprising placing at least one mechanical stimulator on the patient and providing at least one vibrational pulse through the at least one mechanical stimulator, thereby treating the at least one symptom of RLS.

15. A system for treating one or more symptoms of restless leg syndrome (RLS), comprising:
a pair of leggings comprising one or more electrical stimulators and one or more mechanical stimulators arranged on an inner surface thereof;
one or more pulse generators in electrical communication with the one or more electrical stimulators and the one or more mechanical stimulators;
a power source in electrical communication with the one or more pulse generators; and
a mobile device in communication with the pulse generator and comprising at least one processor programmed and/or configured to control one or more characteristics of pulses generated by the pulse generator.

16. The system of claim 15, wherein the power source is removably coupleable with the pair of leggings.

17. The system of claim 16, wherein the power source is a rechargeable battery.

18. The system of claim 15, wherein the at least one processor is programmed and/or configured to generate a user interface on the mobile device configured to allow a patient to control the one or more characteristics of the pulses.

19. The system of claim 18, wherein the user interface allows the patient to control one or more of pulse frequency, pulse width, pulse duration, interstimulus interval, and duration of stimulation.

20. The system of claim 15, wherein the one or more electrical stimulators and one or more mechanical stimulators are arranged such that one or more of the stimulators overlays at least one of a patient's hip, waist, thigh, calf, shin, and/or buttocks.
